(19) United States
(12) Patent Application Publication
(10) Pub. No.: US 2001/0036928 A1
(43) Pub. Date: Nov. 1, 2001

(54) Heterologous boosting immunizations

(76) Inventors: Ronald S. Chamberlain, North Potomac, MD (US); Kari R. Irvine, Washington, DC (US); Steven A. Rosenberg, Potomac, MD (US); Nicholas P. Restifo, Washington, DC (US)

Correspondence Address: MORGAN & FINNEGAN, L.L.P., 345 Park Avenue, New York, NY, 10154-0053, US

(21) Appl. No.: 09/838,987

(22) Filed: Apr. 20, 2001

Related U.S. Application Data
(63) Continuation of application No. 09/171,086, filed on Jan. 22, 1999.
(60) Provisional application No. 60/015,893, filed on Apr. 22, 1996.

(51) Int. Cl. A61K 48/00

(52) U.S. Cl. 514/44

(57) ABSTRACT

This invention describes methods of vaccination for the effective generation of an antigen-specific immune response. More particularly, this invention describes the use of heterologous vaccination vectors for eliciting an enhanced boosting immunization response. Methods of treatment and prevention of diseases using the vaccination schemes of the invention are also provided.

### FIELD OF THE INVENTION

The present invention relates to the field of immunizations and the use of targeted immunotherapy to effect disease onset and/or disease progression. The present invention also relates to human cancer immunotherapy.

### BACKGROUND OF THE INVENTION

Vaccines for cancer, infectious diseases (e.g., HIV) and autoimmune processes represent a major field of current research. The lack of effective vaccination schemes for these complex diseases represents a major obstacle in the generation of an antigen-specific immune response. Accordingly, effective schemes for administration of vaccine protocols are needed. The potential public health impact of the development of new vaccination schemes for cancer, infectious disease and autoimmune disease is enormous.

Vaccinia viruses have been extensively used in humans as a vaccine and its use against smallpox has led to the worldwide eradication of this disease (Moss, B. Science 252:1662-1667, 1991). Vaccina virus is a member of the pox virus family of cytoplasmic DNA viruses. DNA recombination occurs during replication of pox viruses and this has been used to insert DNA into the viral genome. Vaccinia viruses have the advantages of low cost, heat stability and a simple method of administration. Attempts have been made to develop vaccinia virus vectors for the prevention of other diseases.

Several groups have used recombinant vaccina viruses to provide immunizations in human clinical trials as well. Cooney et al immunized 35 healthy HIV seronegative males with a recombinant vaccinia virus expressing the gp160 envelope gene of HIV (Cooney, E.. The Lancet 337:567-572, 1991). Graham et al randomized 36 volunteers to receive either recombinant vaccinia virus containing the gp160 HIV envelope protein or control vaccinia virus (Graham, B. S. et al. J. Infect. Dis. 166:244-252, 1992). Phase I studies using recombinant vaccinia virus recently began in patients with metastatic melanoma using a recombinant virus expressing the p97 melanoma antigen (Estin, C.D. et al Proc. Nat'l Acad. Sci. 85:1052-1056, 1988).

Fowlpox viruses are members of the pox virus family (avipox virus genus) and have also been utilized in the development of vaccines. Fowlpox virus only replicates in avian cells and cannot replicate in human cells. It is a cytoplasmic virus that does not integrate into the host genome but is capable of expression of a large number of recombinant genes in eukaryotic cells. Recombinant fowlpox virus expressing rabies glycoprotein has been used to protect mice, cats and dogs against live rabies virus challenge. Immunization of chickens and turkeys with a recombinant fowlpox expressing the influenza HA antigen protected against a lethal challenge with influenza virus (Taylor, J. et al Vaccine 6:504-508, 1988). Canarypox virus, another member of the avipox genus similar to fowlpox, was safely administered subcutaneously to 25 normal human volunteers at doses up to 10¹¹ infectious doses (Cadox, M. et al The Lancet 339:1429-1432, 1992). In a recent trial sponsored by the NIAID (Protocol 012A: A Phase I safety and immunogenicity trial of live recombinant canarypox-gp160 MN (ALVAC VCP125 HIV-1gp160 MNO in HIV-1 uninfected adults)) patients received recombinant canarypox virus containing the HIV gp160 gene by intramuscular injection with little to no toxicity.

Fowlpox virus thus represents an attractive vehicle for immunization since it can stimulate both humoral and cellular immunity, it can be economically produced in high titers (10⁹ pfu/ml) and yet its inability to productively infect human cells substantially increases the safety of its use, compared to replicating viruses such as vaccina virus, especially in immunocompromised hosts.

Another considerable advantage of fowlpox virus is that there is apparently little or no cross-reactivity with vaccinia virus and thus previously vaccinated humans will not have pre-existing immune reactivity to fowlpox virus proteins.

Many antigens have been associated with disease states or conditions. One of the best characterized group are antigens associated with human cancers. Tumor associated antigens (TAA) which are recognized by T lymphocytes or tumor infiltrating lymphocytes (TIL) and serve as tumor rejection antigens in vivo have been identified and cloned. The identification of TAA has led to the development of novel recombinant and synthetic vectors expressing either the TAA gene or gene product for evaluation as anti-cancer vaccines. These vaccine strategies include immunization with unique TAA peptide epitopes mixed with adjuvants such as incomplete Freund's adjuvant (“IFA”) or bacillus calmette guerin (“BCG”), intramuscular or “gene gun” immunization with plasmid DNA vaccines encoding the gene for a TAA, immunization with whole TAA protein vaccines, or immunization with recombinant viral or bacterial vaccines containing the gene for a TAA. Many of these approaches have been shown to be effective in eliciting a TAA-specific in vivo CTL response, as well as generating protective or active immunotherapeutic responses in experimental animal systems. However, the effect of repetitive or “boosting” vaccinations on the generation of such an immune response has not been previously examined.

Furthermore, because antitumor immune responses appear to be predominantly cell-mediated responses, the design of vaccination schemes that lead to the generation of cytotoxic lymphocytes specific for tumor associated antigens are needed for effective immunotherapy against cancer.

Therefore, it is an object of the present invention to develop a novel vaccination scheme capable and generating high levels of cytotoxic T lymphocytes (“CTL”)

It is another object of the invention to provide heterologous boosting immunotherapy for diseases including cancer, infectious disease and autoimmune disease.

It is yet another object of the invention to provide a vaccination protocol capable of generating therapeutically effective anti-tumor antibodies against tumor associated antigens (“TAAs”). Such a protocol is designed to immunize a patient against cancer.

### SUMMARY OF THE INVENTION

The present invention relates to methods for generating an antigen-specific immune response capable of preventing and/or treating disease. More specifically, the present invention relates to the use of priming and boosting with two different recombinant vectors (heterologous boosting) for the generation of CTL. The present invention relates to the use of multiple different DNA vectors carrying genes encoding one or more antigens for generating a strong cytotoxic T lymphocyte response to said antigen. The use of different vectors and the same antigen gene(s) for immunization and boosting phases of vaccination provides a novel method for eradication of disease.

The present invention also relates to human cancer immunotherapy and the use of heterologous immunizations for treatment of cancers in humans. The immunotherapy methods of the present invention relates to the use of at least two different recombinant vectors expressing the same tumor-associated antigen for immunizing and boosting vaccinations for active treatment of malignant disease. The method mediates powerful CTL responses and anti-tumor immunity.

### BRIEF DESCRIPTION OF THE DRAWINGS

These and other objects, features and many of the attendant advantages of the invention will be better understood upon a reading of the following detailed description when considered in connection with the accompanying drawings:

FIG. 1.: Shows prolonged survival of tumor-bearing animals after immunizing and boosting with different recombinant vectors.

FIG. 2: In vivo, secondary CTL responses in mice immunized with different homologous and heterologous vaccination regimes. CT26.WT (β-gal-,O) and CT26.CL25 (β-gal+, ●) served as targets. “E:T Ratio” represents the Effector to Target ratio. Experiment was repeated seven times with identical results.

FIG. 3A and FIG. 3B: Naive BALB/c mice were vaccinated with either no immunogen (None), 10 μg of β-gal DNA intradermally with the gene gun (DNA), 10⁷ PFU of rVV expressing β-gal (VJS6) intravenously, or 10⁷ PFU or rFPV.bg40k (FPV) intravenously. Twenty-one days later, each group of mice (two/group) was boosted with the same amount of each immunogen to compare all heterologous and homologous immunization regimens. On the day of the boost and eight days following the boost, sera was harvested and assayed for antibody reactivity in ELISA against β-gal protein (FIG. 3A). Sera from mice taken the day of the boost (twenty-one days following the initial immunization) was tested in ELISA against wild-type VV (left panel) or wild-type FPV (right panel). Serum titers to either β-gal protein, VV-WT, or FPV-WT were calculated using the dilution observed at an optical density of 0.3.

FIG. 4: Western Blot of purified β-gal protein, VV-WT, FPV-WT using serum samples from mice immunized with VJS6, FPV.bg40 and pCMV/β-gal DNA. Mice were immunized one time with either 10 μg of β-gal DNA intradermally with the gene gun (left panel), 10⁷ PFU or rVV (VJS6) intravenously (middle panel), or 10⁷ PFU of rFPV.bg40k intravenously (right panel). Twenty-one days later serum was harvested and tested by Western blots at a 1:200 dilution against nitrocellulose blots of 5 μg of β-gal protein (Lanes 2, 5, and 8), 6.6×10⁶ PFU of VV-WT (Lanes 3, 6, and 9), or 2×10⁷ PFU of FPV-WT (Lanes 4, 7, and 10). The blots were then washed and then incubated with horseradish peroxidase-conjugated sheep anti-mouse IgG F(ab′)₂ fragments (1:1000) (Amersham International, Amersham, UK) to visualize antibody binding. Bound immunoglobulin was then detected by incubating the blots for approximately 3 minutes in 3,3′-diaminabenzidine tetrahydrochloride (DAB, Sigma, St. Louis, Miss.) dissolved in dH₂O. The reaction was stopped by washing for five minutes with dH₂O.

### DETAILED DESCRIPTION OF THE INVENTION

The present invention relates to methods of vaccination for the effective generation of an antigen-specific immune response. In particular, the present invention. relates to therapeutic methods of immunotherapy for treatment of disease and thus, prolonged survival in diseased patients. Specifically, the present invention relates to heterologous boosting immunizations for the generation of Cytotoxic T Lymphocytes (“CTL”). The present invention also relates to heterologous boosting immunizations for human cancer immunotherapy for the treatment of cancer patients.

The present invention provides a method for inducing an immunological response in a mammal comprising a first step of inoculating the mammal with a recombinant vaccination vector and a second step of inoculating the mammal with a boosting immunization comprising a second recombinant vaccination vector different from the vector administered in the first step. The vaccination vectors of the present invention comprise viral vectors or plasmid DNAs and one or more genes encoding antigens specifically associated with a particular disease state. Although different vaccination vectors are utilized in step one and step two of the method both vaccination vectors encode at least one common antigen.

Any recombinant vector may be utilized in the present invention, as many are known in the art (Baxby et al. Vaccine, 10:8-9, 1992; Moss et al. Science, 252:1662-1667, 1991; Irvine et al., Sem. Canc. Biol., 6:337-347, 1995. The vector to be used is preferably one that does not integrate with the host organism but effectively expresses the heterologous genes carried on the vector. Recombinant viral vectors used in the present invention.

The recombinant vector has incorporated into its genome a gene encoding an antigen associated with a disease. Optionally, the recombinant vector may also have one or more genes encoding one or more immunostimulatory molecules. A host cell infected with the recombinant vector expresses both the antigen(s) associated with a disease and may optionally also express immunostimulatory molecule(s). Both the antigen and the immunostimulatory molecule may be expressed at the cell surface or may be actively secreted by the host cell.

The priming dose of an antigen results in the activation and expansion of clonotypes capable of recognizing a particular peptide antigen presented in the context of its restricting MHC molecule. Boosting immunization of the present invention, using a different vector than the priming dose leads to strong expansion of the secondary CD8+ T cell population specific for the heterologous antigen. In particular, the up-regulation of the immune response leads to an increase in antigen-specific cytotoxic lymphocytes which are able to kill or inhibit the growth of a disease-causing agent or a diseased cell.

The present invention relates to a “boosting” vaccination strategy that elicits both an enhanced antigen specific CTL and antibody response, while at the same time generating a more therapeutic antigen response. Boosting with a different vector strongly enhances the ability of the recipient mammal to generate antigen specific CTL and antibody responses, thereby leading to the elicitation of a therapeutic response.

In some cases it may be beneficial to make a recombinant vector comprising more than one antigen of interest for the purpose of having a multivalent vaccine. The recombinant vector of the present invention comprises one or more nucleic acid sequences encoding one or more antigens or immunodominant epitopes of the antigens. optionally one or more nucleic acid sequences encoding one or more immunostimulatory molecules may also be carried on the recombinant vector for the purpose of enhancing immune response against the antigen associated with the disease. For example, the recombinant vector may comprise a viral genome or portions thereof, and the nucleic acid sequence encoding an antigen such as, for example, GP120 (from HIV), MART-1, MAGE-1 or Hep B surface antigen.

In one embodiment of the present invention, the treatment of cancer is addressed. In this method, the recombinant vectors used express one or more tumor antigens. optionally, genes encoding cytokines (TNF-α, IFN-γ, GM-CSF, IL-10 and IL-2), restriction elements (class 1 α-chains and β₂ m), and co-stimulatory and accessory molecules (B7-1, B7-2 and ICAM-1 and the like) alone and in a variety of combinations may also be included in the vaccination vector. Simultaneous production of an immunostimulatory molecule and one or more TAAs at the site of virus replication/infection enhances the generation of specific effector molecules, thereby enhancing the therapeutic effect of the present invention. The insertion of costimulatory molecules and/or cytokine genes may also be beneficial in treatment of established metastases.

### Viral Vectors

Viral vectors may be used as recombinant vectors in the present invention, wherein a portion of the viral genome is deleted to introduce new genes without destroying infectivity of the virus. The viral vector of the present invention is a nonpathogenic virus. In one embodiment the viral vector has a tropism for a specific cell type in the mammal. In another embodiment, the viral vector of the present invention is able to infect professional antigen presenting cells such as dendritic cells and macrophages. In yet another embodiment of the present invention, the viral vector is able to infect any cell in the mammal. The viral vector may also infect tumor cells.

Viral vectors used in the present invention include but is not limited to Poxvirus such as vaccinia virus, avipox virus, fowlpox virus and a highly attenuated vaccinia virus (Ankara or MVA), retrovirus, adenovirus, baculovirus and the like.

Expression vectors suitable for use in the present invention comprise at least one expression control element operationally linked to the nucleic acid sequence. The expression control elements are inserted in the vector to control and regulate the expression of the nucleic acid sequence. Examples of expression control elements are well known in the art (Ausubel et al., (1987) in “Current Protocols in Molecular Biology”, John Wiley and Sons, New York, N.Y.) and include, for example, the lac system, operator and promoter regions of phage lambda, yeast promoters and promoters derived from polyoma, adenovirus, retrovirus or SV40. Additional preferred or required operational elements include, but are not limited to, leader sequence, termination codons, polyadenylation signals and any other sequences necessary or preferred for the appropriate transcription and subsequent translation of the nucleic acid sequence in the host system. It will be understood by one skilled in the art the correct combination of required or preferred expression control elements will depend on the host system chosen. It will further be understood that the expression vector should contain additional elements necessary for the transfer and subsequent replication of the expression vector containing the nucleic acid sequence in the host system. Examples of such elements include, but are not limited to, origins of replication and selectable markers. It will further be understood by one skilled in the art that such vectors are easily constructed using conventional methods (Ausubel et al., (1987) in “Current Protocols in Molecular Biology”, John Wiley and Sons, New York, N.Y.) or commercially available.

The vaccinia virus genome is known in the art and it is composed of a Hind F13L region, TK region, and an HA region. The recombinant vaccinia virus has been used in the art to incorporate an exogenous gene for expression of the exogenous gene product (Perkus et al. Science 229:981-984, 1985; Kaufman et al. Int. J. Cancer 48:900-907, 1991; Moss Science 252:1662, 1991).

A general strategy for construction of vaccinia virus expression vectors is known in the art (Smith and Moss Bio Techniques Nov/Dec, p. 306-312, 1984; U.S. Pat. No. 4,738,846). A gene encoding an antigen associated with a disease may be incorporated into the Hind F13L region, or alternatively, incorporated into the TK region of recombinant vaccinia virus vector. Likewise, a gene encoding an immunostimulatory molecule may be incorporated into the Hind F13L region or the TK region of recombinant vaccinia virus vector.

Sutter and Moss (Proc. Nat'l. Acad. Sci U.S.A. 89:10847-10851, 1992) and Sutter et al. (Virology 1994) disclose the construction and use as a vector, the non-replicating recombinant Ankara virus (MVA, modified vaccinia Ankara) which may be used as a viral vector in the present invention. Alternatively, the vector described by Baxby et al. (Vaccine 10:8-9, 1992) may be used as a viral vector in the present invention.

### Antigens Associated With Specific Diseases

The method of the present invention is effective in treating or preventing disease. Many diseases have specific antigens associated with the disease state. Such antigens or immunodominant epitopes of these antigens are crucial to immune recognition and ultimate elimination or control of the disease in a patient. Such antigens are referred to in the art as protective antigens.

The method of the present invention may be used to treat any disease wherein a specific antigen or group of antigens is associated with the disease state. The immunotherapy method of the present invention may be used to treat diseases, for example, human acquired immune deficiency syndrome, HIV, bacterial infections, viral infections, autoimmune diseases and cancers. Specific examples of cancer types include but are not limited to melanoma, metastases, adenocarcinoma, thyoma, lymphoma, sarcoma, lung cancer, liver cancer, colon cancer, non-Hodgkins lymphoma, Hodgkins lymphoma, leukemias, uterine cancer, breast cancer, prostate cancer, ovarian cancer, cervical cancer, bladder cancer, kidney cancer, pancreatic cancer and the like.

The term melanoma includes, but is not limited to, melanomas, metastatic melanomas, melanomas derived from either melanocytes or melanocytes related nevus cells, melanocarcinomas, melanoepitheliomas, melanosarcomas, melanoma in situ, superficial spreading melanoma, nodular melanoma, lentigo maligna melanoma, acral lentiginous melanoma, invasive melanoma or familial atypical mole and melanoma (FAM-M) syndrome. Such melanomas in mammals may be caused by, chromosomal abnormalities, degenerative growth and developmental disorders, mitogenic agents, ultraviolet radiation (UV), viral infections, inappropriate tissue expression of a gene, alterations in expression of a gene, and presentation on a cell, or carcinogenic agents. The aforementioned cancers can be assessed or treated by methods of the present invention. In the case of cancer, a gene encoding an antigen associated with the cancer is incorporated into the recombinant virus genome or portion thereof along with a gene encoding one or more immunostimulatory molecules. The antigen associated with the cancer may be expressed on the surface of a cancer cell, may be secreted or may be an internal antigen. In one embodiment the antigen associated with the cancer is a tumor associated antigen (TAA) or portion thereof. Examples of TAA that may be used in the present invention include but are not limited to melanoma TAAs which include but are not limited to MART-1 (Kawakami et al. J. Exp. Med. 180:347-352, 1994), MAGE-1, MAGE-3, GP-100, (Kawakami et al. Proc. Nat'l. Acad. Sci. U.S.A. 91:6458-6462, 1994), CEA, TRP-1, TRP-2, P-15, and tyrosinase (Brichard et al. J. Exp. Med. 178:489, 1993) and the like.

The nucleotide sequence of the MAGE-3 gene is disclosed in Gaugler et al. (J. Exp. Med. 179:921-930, 1994). MAGE-3 is expressed on many tumors of several types, such as melanoma, head and neck squamous cell carcinomas, lung carcinoma and breast carcinoma but not in normal tissues except for testes. The approximately 1.6 Kilobase (kb) cDNA of MART-1 was cloned into a vector and the resulting plasmid, deposited with the American Type Culture Collection (ATCC Deposit Number 75738). The cloning of MART-1 is disclosed in Kawakami et al. (J. Exp. Med. 180:347-352, 1994) and U.S. patent application Ser. No. 08/231,565 (filed Apr. 22, 1994).

Alternatively, the TAA may be CA-19-A (pancreatic cancer), CA-125 (ovarian cancer), PSA (prostate cancer), erb-2 (breast cancer, CA-171A) and the like (Boon et al. Ann. Rev. Immunol 12:337, 1994).

The present invention is in no way limited to the genes encoding the above listed TAAs. Other TAAs are known to the skilled artisan and may be readily prepared by known methods, such as those disclosed in U.S. Pat. No. 4,514,506.

Genes encoding an antigen associated with a disease wherein the disease is caused by a pathogenic microorganism include viruses, bacteria and protozoans. Examples of viral agents include HIV (GP-120, p17, GP-160 antigens), influenza (NP, HA antigen), herpes simplex (HSVdD antigen), human papilloma virus, equine encephalitis virus, hepatitis (Hep B Surface Antigen) feline leukemia virus, canine distemper, rabies virus, and the like. Pathogenic bacteria include but are not limited to Chlamydia, Mycobacteria, Legioniella and the like. Pathogenic protozoans include but are not limited to malaria, Babesia, Schistosoma, Toxiplasma, Toxocara canis, and the like. Pathogenic yeast include Aspergillus, invasive Candida, and the like.

### Costimulation/Accessory Molecules and Cytokines

A gene encoding one or more costimulation/accessory molecules and/or genes encoding an a cytokine may also be incorporated into the genome of a recombinant vaccination vector for use in the method of the present invention. Examples of costimulation molecules include but are not limited to B7-1, B7-2, ICAM1, ICAM-2, LFA-1, LFA-3, CD72 and the like. Examples of cytokines encompassed by the present invention include but are not limited to IL-2, IL-1, IL-3 through IL-9, IL-11, IL-13 through IL-15, G-CSF, M-CSF, GM-CSF, TNFα, IFNα, IFNγ, IL-10, IL-12, regulated upon activation, normal T expressed and presumably secreted cytokine (RANTES), and the like. Examples of chemokines encompassed by the present invention include but are not limited to CTAP III, ENA-78, GRO, I-309, PF-4, IP-10, LD-78, MBSA, MIP-1α, MIP1B and the like.

The IFNγ construct, TNFα construct, GM-CSF construct and ICAM-1 construct are described in Davidson et al (Nucleic Acid Research 18 (No. 14):4285-4286, 1991).

The IL-2 gene of the present invention was made as disclosed by Taniguchi et al (Nature 302:305, 1983). In one embodiment the entire IL-2 gene as disclosed in Taniguchi et al is incorporated into the TK gene sequence of vaccinia virus. The promotor sequence for the IL-2 construct of the present invention is made up of the P synthetic late promotor as disclosed in Davidson et al (Nucleic Acid Research 18 (14:4285-4286, 1991).

Also encompassed in the present invention is the use of a chimeric gene containing a pox virus promotor region linked to the coding segment of one or more foreign genes encoding an antigen(s) associated with a disease and the coding segment of one or more foreign genes encoding an immunostimulatory molecule(s). The chimeric genes are then incorporated into the pox virus genome by homologous recombination in cells that have transfected with a plasmid vector containing the chimeric gene and infected with the pox virus.

Co-stimulatory molecules of the B7 family (namely B7.1, B7.2, and possibly B7.3) represent a more recently discovered, but important group of molecules. B7.1 and B7.2 are both member of the Ig gene superfamily. These molecules are present on macrophages, dendritic cells, monocytes, i.e., antigen presenting cells (APCs). If a lymphocyte encounters an antigen alone, with co-stimulation by B7.1, it will respond with either anergy, or apoptosis (programmed cell death); if the co-stimulatory signal is provided it will respond with clonal expansion against the target antigen. No significant amplification of the immune response against a given antigen occurs without co-stimulation (June et al. (Immunology Today 15:321-331, 1994); Chen et al. (Immunology Today 14:483-486); Townsend et al. (Science 259:368-370)). Freeman et al. (J. Immunol. 143:2714-2722, 1989) report cloning and sequencing of B7.1 gene. Azuma et al. (Nature 366:76-79, 1993) report cloning and sequencing B7.2 gene.

In one embodiment the B7.1 gene may be inserted into the Hind F13L region of the vaccinia virus, with the β-gal placed in the TK region. The construct for B7.2 and B7.1/B7.2 in conjunction with a tumor antigen are prepared in the same fashion as B7.1. In another embodiment the B7 gene is inserted into the TK region of vaccinia virus and the gene encoding β-gal inserted in the Hind F13L region of the vaccinia virus.

The present invention also encompasses methods of treatment or prevention of a disease. In the method of treatment, the administration of the recombinant vectors of the invention may be for either “prophylactic” or “therapeutic” purpose. When provided prophylactically, the recombinant vector of the present invention is provided in advance of any symptom. The prophylactic administration of the recombinant virus serves to prevent or ameliorate any subsequent infection or disease. When provided therapeutically, the recombinant virus is provided at (or after) the onset of a symptom of infection or disease. Thus the present invention may be provided either prior to the anticipated exposure to a disease-causing agent or after the initiation and/or progression of the infection or disease.

The identification of tumor-specific antigens allows for the development of targeted antigen-specific vaccines for cancer therapy. Insertion of a tumor antigen gene in the genome of multiple different viral vectors provides a powerful system to elicit specific immune response for prevention in patients with an increased risk of cancer development (preventive immunization), prevention of disease recurrence after primary surgery (anti-metastatic vaccination), or as a tool to expand the number of CTL in vivo, thus improving their effectiveness in eradication of diffuse tumors (treatment of established disease). Finally, the method of the present invention may elicit an immune response in a patient that is enhanced ex vivo prior to being transferred back to the tumor bearer (adoptive immunotherapy).

The term “unit dose” as it pertains to the inoculum refers to physically discrete units suitable as unitary dosages for mammals, each unit containing a predetermined quantity of recombinant virus calculated to produce the desired immunogenic effect in association with the required diluent. A unit dose of a viral vector will vary depending upon the virus selected for use. Generally, a unit dose comprises a viral titer in the range of 10⁶-10¹⁰ plaque forming units (PFU). When other DNA vectors are used, 1-1000 μg is the preferred range for a unit dose. The unit dose may be the same for priming and boosting immunizations or it may be desired to alter the quantity of recombinant vector provided in the boosting phase as compared to the initial priming dose. The unit dose of an inoculum of this invention is dictated by and dependent upon the unique characteristics of the recombinant vectors and the particular immunologic effect to be achieved, as is well-recognized by the skilled artisan.

In providing a mammal with multiple recombinant vectors, preferably a human, the dosage of administered recombinant vectors will vary depending upon such factors as the mammal's age, weight, height, sex, general medical condition, previous medical history, disease progression, tumor burden and the like.

The inoculum is typically prepared as a solution in tolerable (acceptable) diluent such as saline, phosphate-buffered saline or other physiologically tolerable diluent and the like to form an aqueous pharmaceutical composition. Adjuvants known in the art are also suitable for the preparation of a unit dose.

The route of inoculation may be intravenous (I.V.), intramuscular (I.M.), subcutaneous (S.C.), intradermal (I.D.) intraperitoneal (I.P.) and the like, which results in eliciting a protective response against the disease causing agent. A priming dose is administered at least once, and may be provided in multiple doses. Boosting doses comprising a different vector encoding the same antigen as the priming dose follow and may be administered in one or more unit doses.

The recombinant vector can be introduced into a mammal either prior to any evidence of cancers such as melanoma or to mediate regression of the disease in a mammal afflicted with a cancer such as melanoma. Examples of methods for administering the vector into mammals include, but are not limited to, exposure of cells to the recombinant virus ex vivo, or injection of the recombinant vector into the affected tissue or intravenous S.C., I.D., I.P. or I.M. administration of the vector. Alternatively the recombinant vector or combination of recombinant vectors may be administered locally by direct injection into the cancerous lesion or topical application in a pharmaceutically acceptable carrier. The quantity of recombinant viral vector, carrying the nucleic acid sequence of one or more TAAs to be administered is based on the titer of virus particles. A preferred range of the immunogen to be administered is 10⁵ to 10¹⁰ PFU per dose, preferably in a human.

After immunization the efficacy of the vaccine can be assessed by production of antibodies or immune cells that recognize the antigen, as assessed by specific lytic activity or specific cytokine production or by tumor regression. One skilled in the art recognizes the conventional methods to assess the aforementioned parameters. If the mammal to be immunized is already afflicted with cancer or metastatic cancer, the vaccine may be administered in conjunction with other therapeutic treatments.

In one method of treatment, autologous cytotoxic lymphocytes or tumor infiltrating lymphocytes may be removed from the patient with cancer as disclosed in U.S. Pat. No. 5,126,132 and U.S. Pat. No. 4,690,915. The lymphocytes are grown in culture and antigen specific lymphocytes expanded by culturing in the presence of the recombinant vectors of the present invention. The antigen specific lymphocytes are then reinfused back into the patient.

The present invention also encompasses combination immunotherapy. By combination therapy is meant that the recombinant vector containing one or more genes encoding one or more antigens associated with one or more disease agents and, optionally, one or more genes encoding immunostimulatory molecules is administered to the patient in combination with other exogenous immunomodulators or immunostimulatory molecules, chemotherapeutic drugs, antibiotics, antifungal drugs, antiviral drugs and the like alone or in combination thereof. Examples of other exogenously added agents include exogenous IL-2, IL-6, IL-10, IL-12, GM-CSF, interferon, IL-10, tumor necrosis factor, RANTES (Promega, G5661), cyclophosphamide, and cisplatin, gancyclovir, amphotericin B and the like.

The present invention establishes that a boosting vaccination with a different vaccine vector (“heterologous boosting”) expressing a TAA rather than the same vaccine vector (“homologous boosting”) elicits a more potent TAA-specific primary CTL response. Similar responses were seen in two separate model TAA system, i.e., β-galactosidase, and influenza (A/PR/8/34) nucleoprotein (NP).

Further, the present invention demonstrates that the generation of an antibody and a primary TAA-specific CTL response following vaccination with plasmid DNA encoding a model TAA is enhanced by a boosting vaccination with either rFPV or rVV expressing the TAA, but not with a boosting vaccination of the same DNA plasmid vector.

The present invention also found that the generation of a primary TAA-specific CTL response following vaccination with a rVV expressing a model TAA is enhanced by a boosting vaccination with a rFPV expressing the TAA, but not with a boosting vaccination of the same rVV vector. Antibody responses can be enhanced with both homologous and heterologous vectors.

Further, the generation of a primary TAA-specific CTL response following vaccination with a rFPV expressing a model TAA is enhanced by a boosting vaccination with a rVV expressing the TAA, but not with a boosting vaccination of same rFPV vector. Antibody responses are enhanced with both homologous and heterologous vectors. The generation of a primary TAA-specific CTL response following vaccination with rAdeno expressing a model TAA can be enhanced by a boosting vaccination with either a rVV or rFPV expressing the TAA, but not with a boosting vaccination with the same rAdeno vector. The present invention also demonstrates that boosting responses which elicit enhanced CTL responses correlate with prolonged survival in tumor-bearing animals.

The foregoing description of the details of the present invention fully reveal the general nature of the invention and others can, by applying current knowledge, readily modify and/or adopt for various applications specific embodiments without departing from the generic concept. Therefore, such adaptations and modifications are intended to be comprehended within the meaning and range of equivalents of the disclosed embodiments.

All articles, books, and patents referred to herein are incorporated, in toto, by reference.

The present invention is described in the following experimental detailed section, which sets forth specific examples to aid in the understanding of the invention, and should not be construed to limit the invention in any way. The following section describes some of the standard materials and methods used in the Examples which follow.

Tumor cell lines and animals. CT26.WT is a clone of the N-nitroso-N-methylurethrane induced BALB/c (H-2^{d}) undifferentiated colon carcinoma. Following transduction with a retrovirus encoding the lacZ gene. CT26.WT was subcloned to generate the β-gal expressing cell line CT26.CL25 (Wang et al. J. Izmnunol. 154(9):4685-4692, 1995). Cell lines were maintained in RPMI 1640, 10% heat inactivated FCS (Biofluids, Rockville, Md.), 0.03% L-glutamine, 100 μg/ml streptomycin, 100 U/ml penicillin and 50 μg/ml gentamicin sulfate (NIH Media Center). CT26.CL25 was maintained in media containing 400 or 800 μg/ml G418 (GIBCO, Grand Island, N.Y.). Female BALB/c mice, 6 to 10 wk old, were obtained from the Animal Production Colonies, Frederick Cancer Research Facility, national Institutes of Health (Frederick, Md.).

Plasmid preparations and Gene Gun Delivery of DNA. A plasmid encoding the Escherichia coli lacZ gene (pCMV/β-gal) under the control of the human CMV intermediate-early promotor, designated pCMV/β-gal was kindly provided by J. Haynes (Agracetus, Middleton, Wis.). A plasmid expressing the nucleoprotein from influenza A virus (A/PR/8/34) also under the control of the CMV promotor was used as a control vector in this study. Closed circular plasmid DNA was isolated using Wizard maxipreps DNA purification kits (Promega Corp, Madison, Wis.). Plasmid DNA and gold were coprecipitated by the addition of 200 μl of 2.5 M Cacl₂ during vortex mixing as previously described (Fuller et al., AIDS Res. Hum. Retrovir, 10(11):1433, 1994). DNA-coated gold particles were delivered into abdominal epidermis using the hand-held helium driven device Accell® gene delivery system (kindly provided by Geniva, Middleton, Wis.). Each animal received 10 non-overlapping deliveries per immunization at a pressure of 400 psi of helium.

Recombinant viruses. The recombinant vaccinia virus (rVV) vaccine, VJS6, was engineered such that the E.coli lacZ gene encoding β-gal, was under the control of the early/late VV 7.5K promoter from plasmid pSC65 (Bronte et al. J. Immunol., 154(10):5282-5292, 1995). The rVV, V69, was similarly constructed such that the gene encoding for nucleoprotein from influenza A (A/PR/8/34) was under the control of the early/late 7.5K promoter from plasmid PSC65 (V69) (Smith et al., Virology, 160:336-345, 1987). The recombinant stocks were initially propagated in the BSC-1 monkey kidney cell line to create a crude lysate which was then further purified over a sucrose cushion. The recombinant fowlpox viral (rFPV) vaccine used in these studies (FPV.bg40k) contains the E.coli lacZ gene under control of the vaccinia virus 40K promoter inserted into the BamHI region of the FPV genome as previously described (Therion Biologies Corp., Cambridge, Mass.) (Wang et al., J. Immunol., 154(9):4685-4692, 1995).

### EXAMPLE 1

### Boosting with Heterologous Vectors Prolong Survival of Tumor-bearing Mice

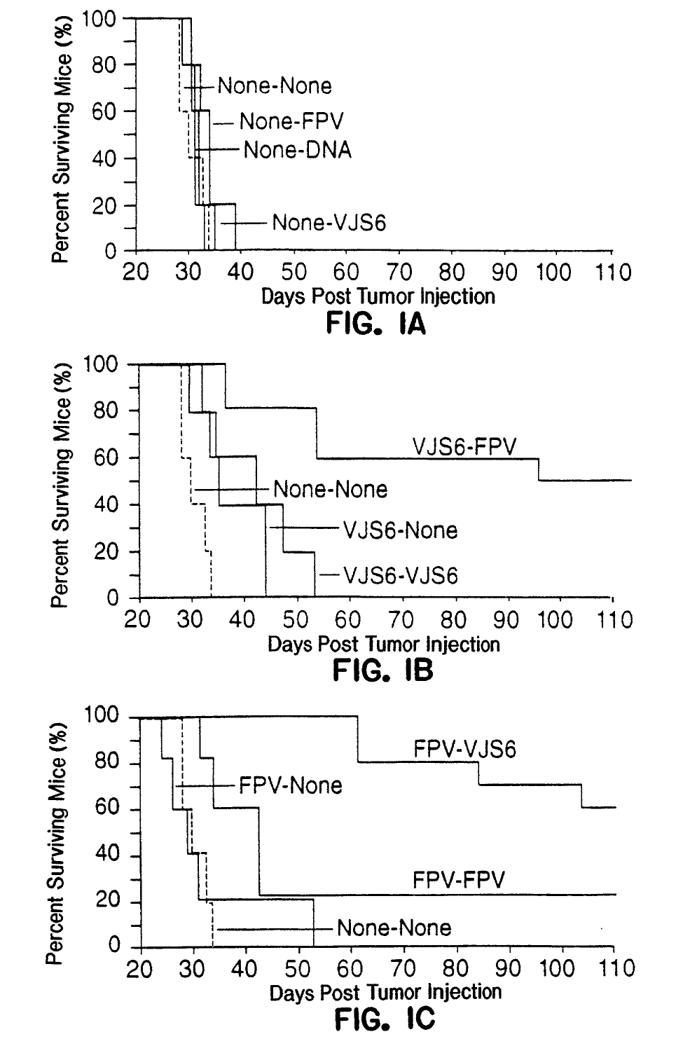

To compare the effect of repetitive immunization of the recombinant vaccine vectors on tumor growth, long-term survival studies were performed (FIG. 1). BALB/c mice were challenged intravenously with 10⁵ CT26.CL25 tumor cells to establish pulmonary metastases (Rao et al. J. Immunol., 156:3357-3365, 1996). Three days later, groups of mice (ten/group) were primed with either (FIG. 1, Panel A) no immunogen (None) (FIG. 1, Panel B) 10⁷ PFU of rvv expressing β-gal (VJS6) intravenously, (FIG. 1, Panel C) 10⁷ PFU of rFPV expressing β-gal, rFPV.bg40(rFPV) intravenously, (FIG. 1, Panel D) 10 μg of pCMV/β-gal (DNA) intradermally with the gene gun. Seventeen days after tumor inoculation, each group of mice was boosted with the same amount of each immunogen to compare all possible heterologous and homologous immunization strategies and followed for long-term survival. Statistical analysis was performed with Kaplan-Meier survival curves. In FIG. 1, Panel E, mice were administered either no treatment, VJS6, rFPV.bg40 of pCMV/β-gal three days after tumor inoculation and then boosted with pCMV/β-gal DNA fourteen days later. The no treatment group (None-None) is shown in all graphs of FIG. 1 as a control group.

FIG. 1 represents data from one experiment performed identically two times with similar results. Mice initially immunized with VJS6 then received a boosting vaccination with either the FPV.bg40 (FIG. 1, Panel B) or pCMV/β-gal (FIG. 1, Panel E) both exhibited prolonged survival compared to the control unvaccinated group (p₂<0.0001). Mice initially immunized with pCMV/β-gal then received a boosting vaccination with either VJS6 or rFPV.bg40 exhibited prolonged survival compared to the no treatment group (p₂, 0.0001 for both) (FIG. 1, Panel D).

BALB/c mice challenged intravenously with CT26.CL25 (β-gal+) tumor cells were immunized three days later with either no immunogen, pCMV/β-gal, rVV-β-gal (VJS6), or rFPV expressing β-gal (FPV.bg40). Seventeen days after tumor inoculation, each group of mice received a boost with each immunogen to compare all possible heterologous and homologous immunization strategies.

No prolongation of survival was observed in the groups immunized seventeen days following tumor administration with either pCMV/β-gal, VJS6, or FPV.bg40 compared to unimmunized mice (FIG. 1A). Two immunizations with VJS6 prolonged survival compared to unvaccinated mice but this was not statistically different than one immunization three days after tumor challenge (FIG. 1B). Mice that received a boost with a heterologous recombinant viral vector, rFPV.bg40, had a longer survival time compared to mice that received the homologous prime and boost with VJS6 (p₂<0.00001, FIG. 1B) . Indeed, 50% of the heterologously boosted mice survived longer than 110 days. A similar pattern was observed for rFPV immunization (FIG. iC). Mice administered rFPV.bg40 and boosted with the heterologous vector, VJS6, resulted in a significant increase in survival compared to the mice that received two doses of rFPV.bg40 (p₂<.00001); 60% of the mice that received the heterologous combination survived for greater than 100 days (FIG. 1C).

For DNA immunization, a small but significant increase in survival was observed in the group of mice that received a prime and a boost with pCMV/β-gal (p₂=0.0018) (FIG. 1D). Boosting pCMV/β-gal immunization with either heterologous vector, VJS6 or rFPV.bg40, significantly extended longevity compared to the no treatment group (p₂=0.0001) or to single prime of DNA (p₂<.0001) (FIG. 1D). Conversely, boosting with pCMV/β-gal increased the lifespan of mice primed with either VJS6 or rFPV.bg40 compared to mice immunized two times with pCMV/β-gal (p₂<0.0001, FIG. 1E). No statistical difference in survival was observed between mice primed with either VJS6 or rFPV.bg40 boosted with pCMV/β-gal and the groups of mice that received a homologous prime and boost of either rFPV.bg40 or VJS6 (FIG. 1 D&E). Altogether, these data suggested that immunizing and boosting with two different vectors expressing the same TAA prolongs survival of tumor-bearing mice more efficiently than multiple immunizations with the same vector.

### EXAMPLE 2

### In Vvivo Secondary CTL Responses Induced in Mice Immunized and Boosted with Different Vectors Expressing the Same TAA

To determine the effect of the different immunization schema on the induction of an antigen-specific CTL response, mice were immunized with the different heterologous and homologous combinations of the pCMV/β-gal, VJS6 and rFPV.bg40 vaccines.

BALB/c mice were vaccinated with either no immunogen, 10 μg of β-gal DNA intradermally with the gene gun, 10⁷ PFU of rVV (VJS6 or V69) intravenously, or 10⁷ PFU of FPV.bg40k intravenously. Twenty-one days later, each group of mice was boosted with the same amount of each immunogen to compare all heterologous and homologous possibilities. To determine the optimal kinetics of an in vivo secondary CTL response, mice were sacrificed 2, 4, 6, and 8 days after the second vaccination at which time their spleens were removed and CTL lytic reactivity against β-gal without an in vitro stimulation step was assessed in a standard 6-hour ⁵¹Cr release assay. For all other experiments, mice were sacrificed at the optimal time-point, 4 days following the second vaccination and in vivo CTL lytic reactivity was assessed. Pooled serum (2 mice/group) was also taken eight days following the boost to evaluate antibody reactivity of β-gal protein via an ELISA.

⁵¹Cr release assay. Six-hour ⁵¹Cr release assays were performed as previously described (Restifo et al., J. Exp. Mod., 177:265-272, 1993). Briefly, 2×10⁶ target cells were incubated on 0.2 ml of CM labeled with 200 μCi of Na⁵¹ CrO₄ for 90 min. Peptide-pulsed CT26.WT were incubated with 1 μg/ml (approximately 1 μM) antigenic peptide during labeling. Target cells were then mixed with effector cells for 6 h at 37° C. at the effector to target ratios indicated. The amount of ⁵¹Cr released was determined by gamma counting and the percentage of specific lysis was calculated as follows:

% specific lysis=[(experimental cpm−spontaneous cpm)/(maximal cpm−spontaneous cpm)]×100.

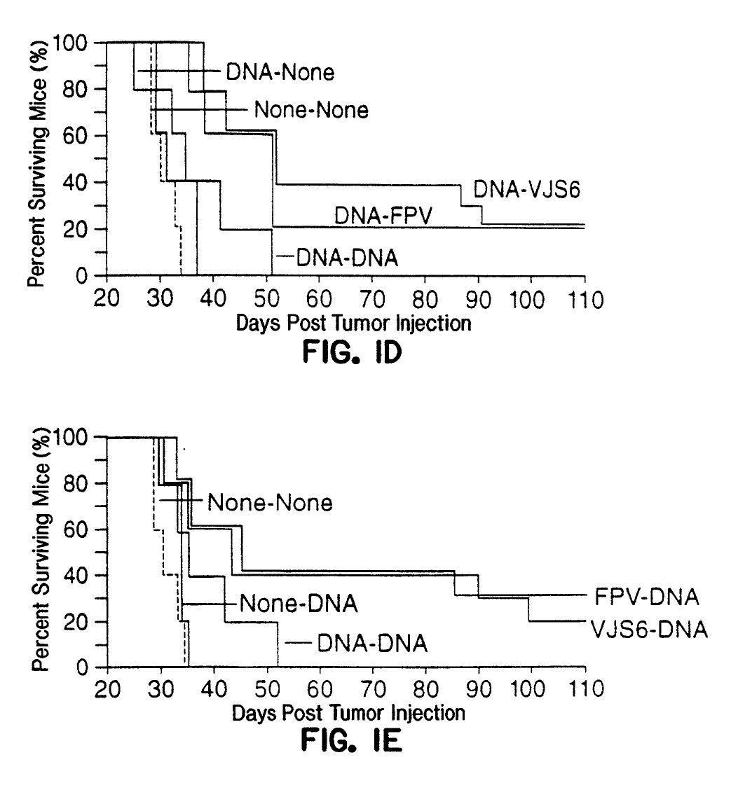

Unprimed mice administered VJS6 or rFPV.bg40 and tested for CTL reactivity four days later failed to induce a lytic response against either CT26.CL25 (β-gal+) or CT26.WT (β-gal−). Mice primed with either VJS6 or rFPV.bg40 and tested twenty-one days later did not elicit β-gal-specific CTL. No CTL activity was observed when mice were immunized and boosted with the same vector, either VJS6 or rFPV.bg40 (FIG. 2). However, boosting the VJS6-primed mice with a different vector, rFPV.bg40, induced antigen-specific CTL (FIG. 2). Mice primed and boosted with rFPV.bg40 also did not induce anti-β-gal CTL. However, rFPV.bg40-primed mice boosted with the heterologous vector, VJS6, elicited antigen-specific CTL (FIG. 2). Mice primed with pCMV/β-gal DNA induced β-gals-pecific CTL only when boosted with either VJS6 or rFPV.bg40 (FIG. 2). The order of this immunization appeared to be important because when either a VJS6 or rFPV.bg40 immunization was followed by a booster with pCMv/β-gal DNA, no lytic activity was observed. Together, these studies suggest that repetitive vaccination with the same vector does not promote the expansion of antigen-specific CTL. However, the immunization strategy using two different recombinant vectors expressing the same antigen does induce enhanced lytic activity.

### EXAMPLE 3

### Augmented Anti-β-gal Antibody Responses were Elicited Following a Boost with any Combination of pCMV/β-gal, rVV or rFPV

To study antigen-specific humoral immunity using the different combinations of the rDNA, rVV and rFPV vaccines, serum samples, harvested eight days following the boost, were tested by ELISA for antibody reactivity against β-gal protein.

Enzyme-linked immunosorbent assay. Serum from immunized mice was collected twenty-one days following the primary immunization and eight days following the final boost to be analyzed for the presence of antibodies against β-gal , wild-type vaccinia virus or wild-type fowlpox virus by ELISA, as previously described (Irvine et al. J. Immunol., 256:238-245, 1996). Specifically, microtiter plates were either dried down overnight at 37° C. in a nonhumidified incubator with 200 ng/well/50 μl of purified β-gal protein (Sigma Chemical Co., St. Louis, Mo.). Alternatively, microtiter plates were coated with either WT-VW (5×10⁵/well/50 μl) or WT-FPV (5×10⁵/well/50 μl) at 4° C. overnight. Incubation of 5% BSA in PBS on each well for 1-h to prevent nonspecific Ab binding was followed by a second 1-h incubation with 50 μl of fivefold dilutions (starting at 1:100) of test sera. After washing with 1% BSA in PBS, horseradish peroxidase-conjugated sheep anti-mouse IgG F(ab′)₂ fragments (1:3000) (Amersham International, Amersham, UK) were added for 1 h at 37° C. to detect antibodies immobilized of the wells. The resulting complex was detected by the chromogen, 0-phenylenediazamine (Sigma Chemical Co.). Absorbance was read on a Titertek Multiskan Plus reader (Flow Laboratories, McLean, Va.) using a 490-nm pore filter.

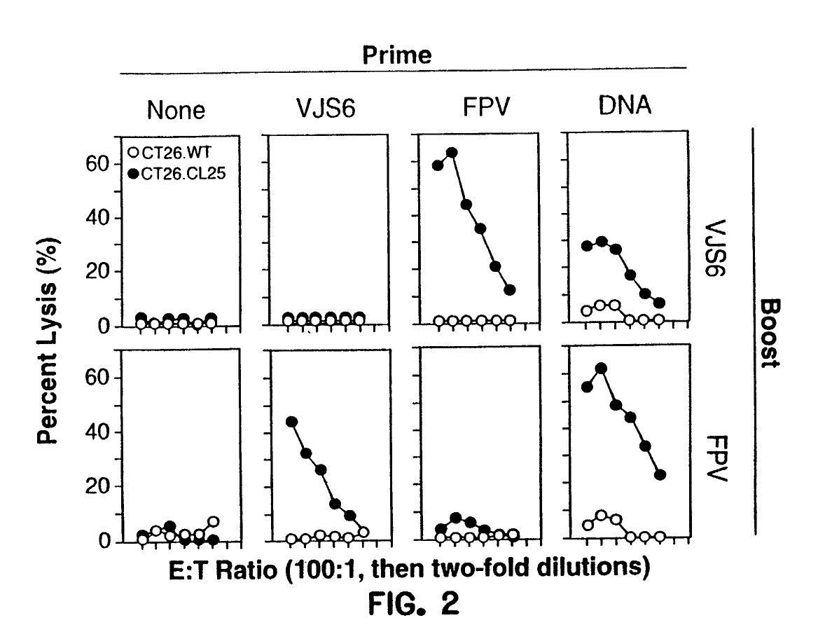

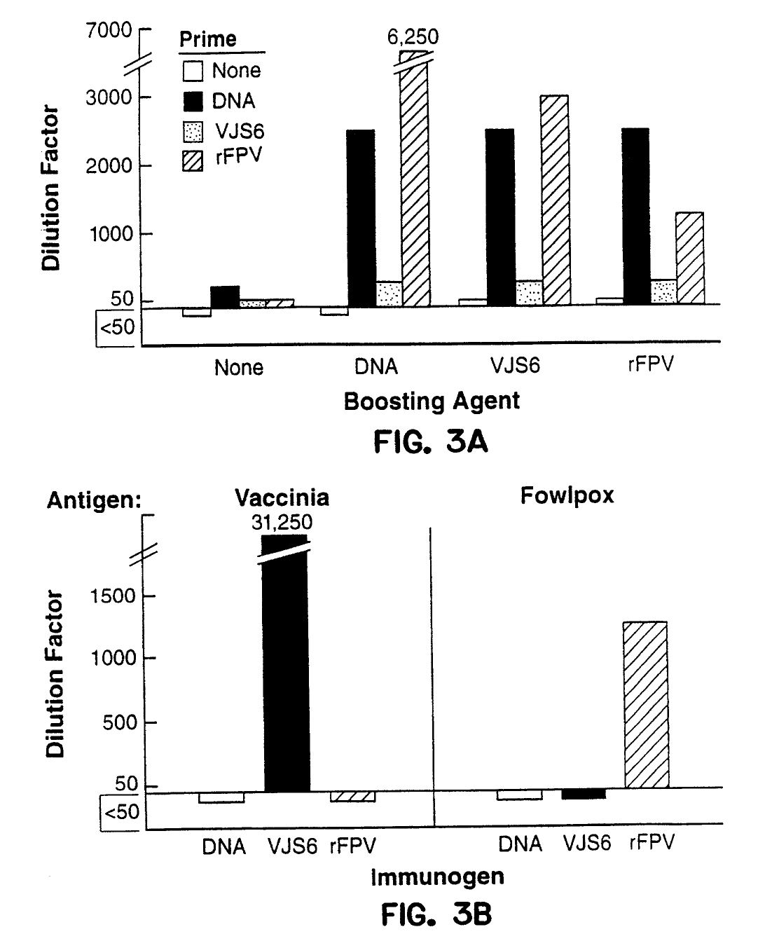

β-gal-specific antibody titers were increased following a primary immunization with VJS6 with boosts of either pCMV/β-gal, VJS6, or rFPV (Titers increased from 1:50 with no boost to 1:250 for each group, FIG. 3A). Following rFPV.bg.40 immunization, β-gal titers were also dramatically boosted by a second immunization with either pCMV/β-gal (Titer=1:6,250), VJS6 (Titer=1:3,000), or rFPV (Titer=1:1,500). β-gal-specific antibody titers were also boosted when either pCMV/β-gal, VJS6, or rFPV were administered as a boost following pCMV/β-gal priming; these ranged from 1:200 to 1:2,500 for each (FIG. 3A). In contrast to CTL activity, an enhancement of the anti-β-gal antibody response was observed regardless of boosting with either a homologous vector or a heterologous vector expressing the same TAA.

### EXAMPLE 4

### Vector-specific, High-titered Antibodies were Induced Following a Single Immunization of Either rVV or rFPV

To characterize vector-specific humoral immunity induced by immunization with either of the pCMV/β-gal, VJS6 or rFPV.bg40 vaccines, serum samples harvested twenty-one days following the primary immunization were tested by ELISA (as described in Example 3) and Western blot for antibody reactivity against wild-type vaccinia virus (VV-WT) or wild-type fowlpox virus (FPV-WT) (FIG. 3B & 4).

Western Blot Analysis. Mouse antiserum obtained 21 days following the primary immunization was tested in a Western blot for reactivity against β-gal protein, WT-VV, and WT-FPV. To this end, 5 μg of β-gal protein, 6.6×10⁶ PFU of VV-WT, and 2×10⁷ PFU of FPV-WT were dissolved in SDS-polyacrylamide gel electrophoresis sample buffer, boiled for 5 min and subjected to electrophoresis using a 6-18% linear gradient SDS-polyacrylamide gel. After electrophoresis, proteins were transferred for 2 h to nitrocellulose paper (0.45 um pore size) at RT at 25V in transfer buffer. The blots were then incubated in PBS containing 5% nonfat dry milk for 1 h at RT. Ten ml of a 1:200 dilution of antiserum in PBS with 2% nonfat dry milk were added to each nitrocellulose strip and incubated for 2 h at room temperature with gentle agitation. After washing the blots with PBS containing 0.5% Tween-20, the blots were incubated with horseradish peroxidase-conjugated sheep anti-mouse IgG F(ab′)₂ fragments (1:1000) (Amersham International, Amersham, UK) to visualize antibody binding. Bound immunoglobulin was then detected by incubating the blots for approximately 3 minutes in 3,3′-diaminabenzidine tetrahydrochloride (DAB, Sigma, St. Louis, Miss.) dissolved in dH₂O. The reaction was stopped by washing for five minutes with dH₂O.

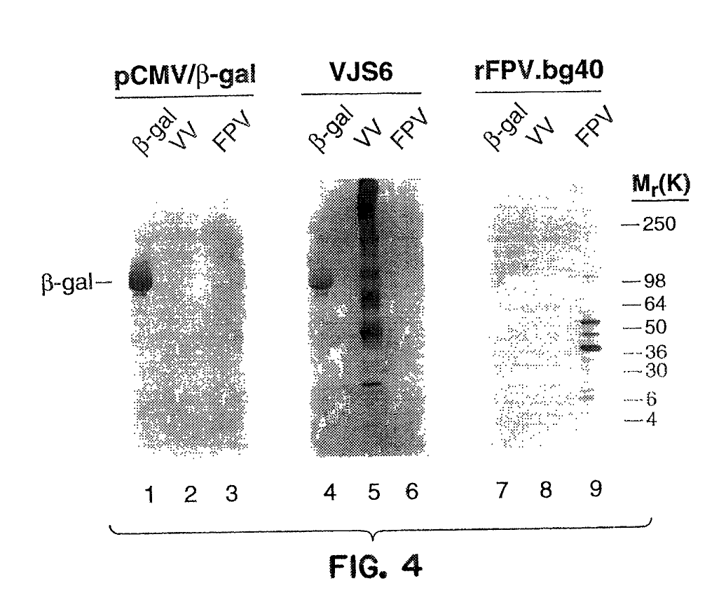

High titers of anti-vaccinia virus antibody were seen by ELISA in the serum from mice primed with VJS6 (Titer=1:31,250), but not in the serum from mice immunized with pCMV/β-gal or rFPV.bg40 (FIG. 3B). Western blot analysis demonstrated that immunization with VJS6 induced antibodies against both a single band of β-gal protein (FIG. 4, Lane 5) and a hundreds of bands of WT-VV (Lane 6), but no reactivity was observed against WT-FPV (FIG. 4, Lane 7). Similarly, titers of anti-fowlpox virus antibodies were only found in the sera of mice primed with rFPV.bg40 (Titer=1:2,250, FIG. 3B). Western blot analysis showed that vaccination with rFPV.bg40 induced antibodies that recognized β-gal protein (FIG. 4, Lane 8), 14-20 bands of WT-FPV (FIG. 4, Lane 10) and no bands of WT-VV (FIG. 4, Lane 9). The antibodies induced by immunization with pCMV/β-gal did not react with either VJS6 or rFPV.bg40 but did recognize β-gal protein both by ELISA and by Western blot analysis (FIG. 3B & 4). These data show that high titers of vector-specific antibodies were induced by immunization with either vaccinia virus or fowlpox viruses.

The anti-vector antibodies may not only play a role in the lack of β-gal specific CTL responses in groups of mice immunized and boosted with the same viral vector (FIG. 2) but may also reduce prolongation of survival in the groups of mice immunized and boosted with the same viral vectors (FIG. 1). In contrast, vaccination strategies using different recombinant vectors expressing the same TAA resulted in no cross-reactive antibodies, enhanced CTL responses and prolonged survival of tumor bearing mice. Thus, this strategy of immunizing and boosting with alternating recombinant vectors may be a more potent means of enhancing an immune response against a desired antigen than repetitive immunizations with the same vector.

### EXAMPLE 5

### Melanoma Patients are Treated with Viral Vectors Expressing TAAs

Large quantities of gmp quality recombinant viral and nonviral vectors expressing the TAAs, human gp100 and MART-1 are produced. In particular, rFPV and rVV that express each of the two aforementioned antigens have been produced (Therion, Inc.). Recombinant adenoviruses expressing TAA are produced (Genzyme, Inc.). In addition, recombinant DNA vectors and Influenza virus vectors expressing gp100 and peptide fragments of gp 100 respectively are produced.

Patients receive either rDNA at 2-8 μg per individual dose, Influenza virus vector or adenovirus (10⁶-10¹¹ pfu/individual). Three to six weeks later patients are boosted heterologously with 10⁶-10¹¹ pfu per individual of either rFPV or rVV. CTL and clinical responses are monitored in these patients. The clinical status of the tumors is evaluated at monthly intervals.

Alternatively, melanoma patients received rVV or rFPV every three weeks at dose ranging from 10⁶-10⁹. Antibody titers against the viral sectors have been measured from the sera of these patients. These patients have received boosting immunizations with heterologous vectors. Patient CTL and clinical responses are being monitored.

What is claimed is:

1. A method for inducing an enhanced immunological response against at least one antigen in a mammal, said method comprising the steps of:
inoculating the mammal with a first recombinant vector comprising a DNA vector and a gene encoding said antigen; and
inoculating the mammal with a boosting immunization with a second recombinant vector comprising a second DNA vector and the gene encoding said antigen.

2. The method according to claim 1, wherein the first recombinant vector comprises a recombinant vaccinia virus vector.

3. The method according to claim 1, wherein the first recombinant vector comprises a recombinant fowlpox virus vector.

4. The method according to claim 1, wherein the first recombinant vector comprises an adenovirus vector.

5. The method according to claim 1, wherein the recombinant vectors further comprise a gene encoding an immunostimulatory molecule.

6. The method according to claim 1, wherein the second recombinant vector comprises a recombinant vaccinia virus vector.

7. The method according to claim 1 wherein the second recombinant vector comprises a recombinant fowlpox virus vector.

8. The method according to claim 1 wherein the second recombinant vector comprises a recombinant adenovirus vector.

9. The method of immunotherapy for treatment of a cancer patient, said method comprising the steps of:
immunizing said patient with an effective amount of a first recombinant vector comprising a first viral vector and a gene encoding a tumor-associated antigen; and
boosting said patient with an effective amount of a second recombinant vector comprising a second viral vector and the gene encoding the tumor-associated antigen.

10. The method according to claim 9, wherein the tumor-associated antigen comprises gp100.

11. The method according to claim 9, wherein the tumor-associated antigen comprises MART-1.

12. The method according to claim 9, wherein the tumor-associated antigen comprises TRP-1.

13. The method according to claim 9, wherein the tumor-associated antigen comprises TRP-2.

14. The method according to claim 9, wherein the recombinant vectors further comprise a gene encoding an immunostimulatory molecule.

15. The method according to claim 9, wherein the first viral vector comprises a vaccinia virus.

16. The method according to claim 9, wherein the first viral vector comprises a fowlpox virus.

17. The method according to claim 9, wherein the first viral vector comprises an adenovirus.

18. The method according to claim 9, wherein the second viral vector comprises a vaccinia virus.

19. The method according to claim 9, wherein the second viral vector comprises fowlpox virus.

20. The method according to claim 9, wherein the second viral vector comprises an adenovirus.
